(19) United States
(12) Patent Application Publication
(10) Pub. No.: US 2023/0005812 A1
(43) Pub. Date: Jan. 5, 2023

(54) IGBT PACKAGE HEATING DISSIPATION STRUCTURE AND MOTOR CONTROLLER APPLYING THE SAME

(71) Applicant: HEFEI JEE POWER SYSTEMS CO., LTD., Hefei (CN)

(72) Inventors: Lei LIU, Hefei (CN); Jie GU, Hefei (CN); Jianhua MAO, Hefei (CN); Yong CHENG, Hefei (CN)

(73) Assignee: HEFEI JEE POWER SYSTEMS CO., LTD., Hefei (CN)

(21) Appl. No.: 17/710,379

(22) Filed: Mar. 31, 2022

Related U.S. Application Data
(63) Continuation of application No. PCT/CN2021/127341, filed on Oct. 29, 2021.

(30) Foreign Application Priority Data
Jul. 5, 2021 (CN) 202110755225.8

(51) Int. Cl. H01L 23/373; H02K 11/33; H05K 1/18; H05K 7/20

(52) U.S. Cl. CPC H01L 23/3735; H02K 11/33; H05K 1/181; H05K 7/20927; H02K 2211/03; H05K 2201/10015; H05K 2201/10166

(57) ABSTRACT

Disclosed are an IGBT package heat dissipation structure and a motor controller applying the same. The IGBT package heat dissipation structure includes a heat dissipation substrate body, heat dissipation fins and electrodes respectively fixed on two sides of the heat dissipation substrate body. Each heat dissipation fin is provided with an inner cavity, a wafer, a first DBC layer and a second DBC layer are arranged in the inner cavity, an electrode is extended through the heat dissipation substrate body, and connected with the wafer through the first DBC layer and the second DBC layer.

### CROSS-REFERENCE TO RELATED APPLICATIONS

This application is a continuation of International Application PCT/CN2021/127341, filed on Oct. 29, 2021, which claims priority to Chinese Patent Application No. 202110755225.8, filed on Jul. 5, 2021, the entire contents of which are incorporated herein by reference.

### TECHNICAL FIELD

The present application relates to insulated gate bipolar transistor (IGBT) heat dissipation technology, in particular to an IGBT package heat dissipation structure and a motor controller applying the same.

### BACKGROUND

IGBT is a core device for energy conversion and transportation, and has wide applications in the fields of rail transit, intelligent power grid, aerospace, electric vehicles and new energy equipment and the like. Since the current carried by the IGBT device in the circuit is large, the power density is high, a good heat dissipation capability is needed to ensure the normal work of the IGBT. The heat dissipation of the IGBT is very important, and poor heat dissipation will seriously affect the service life of the IGBT. However, the heat dissipation performance of the current package structure is not good. Therefore, when the power densities of the existing motor controllers are required to be higher and higher, the IGBT package heat dissipation structure with simple structure and good heat dissipation performance is urgently needed. Due to the important effect of the IGBT for the power electronic devices, the standard for the heat dissipation of the IGBT gets higher and higher.

In the existing IGBT package structure, the wafers are horizontally arranged on a large substrate, which leads to a large packaging volume. The heat dissipation depends on the heat dissipation plate located at the bottom, the heat dissipation efficiency is low and the effect is poor.

### SUMMARY

The present application aims to provide an insulated gate bipolar transistor (IGBT) package heat dissipation structure and a motor controller applying the same. The above technical problems are solved and the following effects are achieved:

the packaging housing of the IGBT and the heat dissipation fins of in the water channel are combined into one, the space is reasonably utilized, and the packaging volume is reduced;

heat dissipation from multi directions is achieved, and the heat dissipation area and heat dissipation efficiency are improved;

the problem of heat dissipation of the IGBT of the controller system after being highly integrated is solved, and the power density of the system is improved.

The technical scheme of the present application is as follows.

An IGBT package heat dissipation structure includes a heat dissipation substrate body and heat dissipation fins and electrodes respectively fixed on two sides of the heat dissipation substrate body; each heat dissipation fin is provided with an inner cavity, a wafer, a first direct bonding copper (DBC) layer and a second DBC layer are arranged in the inner cavity; an electrode is passed through the heat dissipation substrate body, and connected with the wafer through the first DBC layer and the second DBC layer.

In an embodiment, a left side surface of the wafer is connected with a copper layer on a right surface of the first DBC layer by a second welding layer, and a right side surface of the wafer is connected with a copper layer on a left surface of the second DBC layer by a third welding layer.

In an embodiment, the electrode is connected to the copper layer on the right surface of the first DBC layer and the copper layer on the left surface of the second DBC layer to connect with the wafer, so as to supply current to the wafer.

In an embodiment, a copper layer on a left surface of the first DBC layer is connected with a left surface of the inner cavity of the heat dissipation fin by a first welding layer, and a copper layer on a right surface of the second DBC layer is connected with a right surface of the inner cavity of the heat dissipation fin by a fourth welding layer.

In an embodiment, the heat dissipation substrate body is provided with an insulating housing at a position corresponding to the heat dissipation fin, and the electrode is extended into the inner cavity of the heat dissipation fin through the insulating housing.

In an embodiment, the inner cavity of the heat dissipation fin is filled with a heat dissipation material.

In an embodiment, the inner cavity of the heat dissipation fin is an upside down H shape, a U shape, or a trapezoidal shape with a bottom closed.

In an embodiment, a bottom of the inner cavity of the heat dissipation fin is not closed.

A motor controller includes a main housing and a film capacitor, a PCB, a three-phase output module, an IGBT module, and a bus module passing through the main housing, the IGBT module includes the IGBT package heat dissipation structure mentioned above, and a plurality of IGBT package heat dissipation structures share the heat dissipation substrate body.

In an embodiment, a water channel for heat dissipation is provided in the main housing, and the heat dissipation fins of the IGBT module are disposed in the water channel.

The present application has the following advantages.

According to the IGBT package heat dissipation structure provided by the present application, the packaging housing of the IGBT and the heat dissipation fins in the water channel are combined into one, the space is reasonably utilized, the packaging volume is reduced, which is helpful for the miniaturization design of the motor controller.

According to the IGBT package heat dissipation structure provided by the present application, heat dissipation from multi directions can be achieved, the heat dissipation area is large, the heat dissipation efficiency is high, and the effect is good.

The present application is applied to the motor controller, solves the problem of IGBT heat dissipation of the controller system after being highly integrated, and greatly improves the power density of the system.

### BRIEF DESCRIPTION OF THE DRAWINGS

The present application is further described below with reference to the accompanying drawings and embodiments.

FIG. 1 is a schematic structural view of a motor controller.

FIG. 2 is a schematic structural view of the motor controller with a PCB being removed.

FIG. 3 is a schematic structural view of a heat dissipation system of the motor controller.

FIG. 4 is a schematic structural view of an IGBT module.

FIG. 5 is a schematic structural view of the IGBT module, viewed from another angle.

FIG. 6 is a cross-sectional view of the IGBT module.

FIG. 7 is a top view of the IGBT module, viewed from where electrodes locate.

FIG. 8 is an enlarged view of an IGBT package heat dissipation structure.

### DETAILED DESCRIPTION OF THE EMBODIMENTS

### First Embodiment

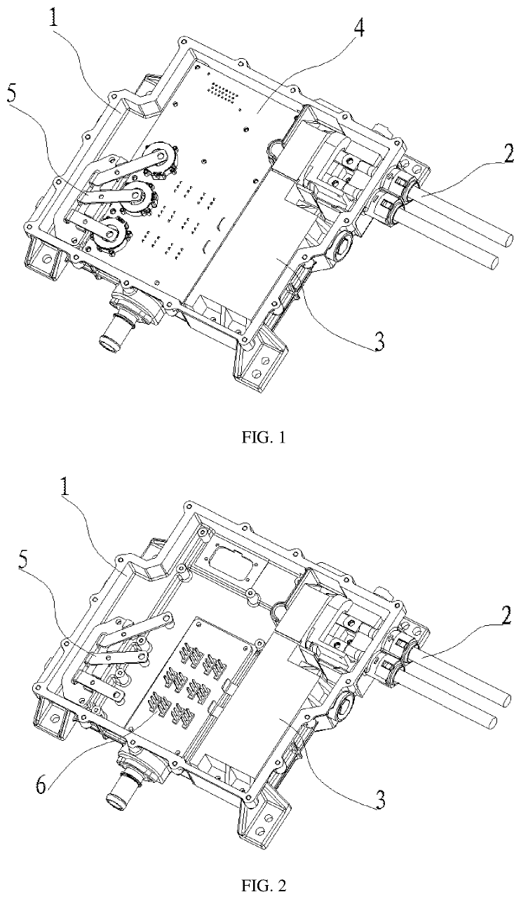

As shown in FIGS. 1 and 2, the motor controller includes a main housing 1, a bus module 2, a film capacitor 3, a printed circuit board (PCB) 4, a three-phase output 5, an insulated gate bipolar transistor (IGBT) module 6, etc. Working current of the motor controller is supplied to the film capacitor 3 from the bus module 2, then to input electrodes of the IGBT module 6 through the PCB 4, and then to the three-phase output 5 from output electrodes of the IGBT module 6 through the PCB 4, and then is supplied to a motor to drive the motor.

As shown in FIG. 3, the principle of a heat dissipation system of the motor controller is as follows: cooling liquid enters a water channel from a water inlet 11 of the main housing 1, and then is in contact with heat dissipation fins 612 of the IGBT module 6, to cool the peripheral portions of the wafers inside the heat dissipation fins 612, and finally the cooling liquid enters a cooling circulation system of a vehicle via the water outlet 12 of the main housing 1. The heat dissipation structure can realize heat dissipation of the IGBT wafers from multi directions, and the heat dissipation efficiency is greatly improved.

### Second Embodiment

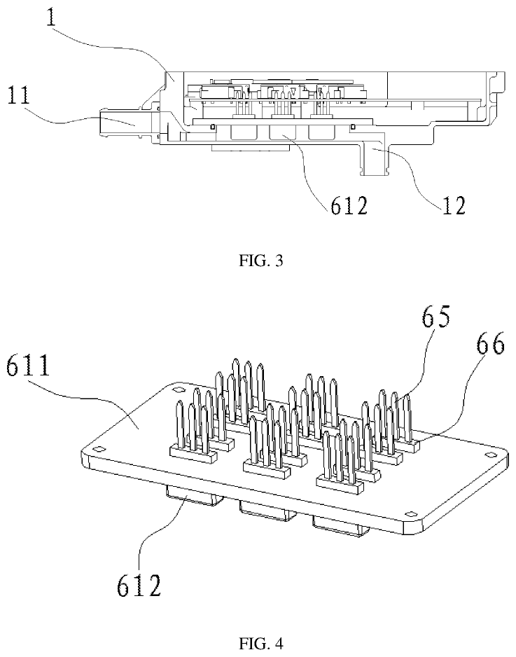

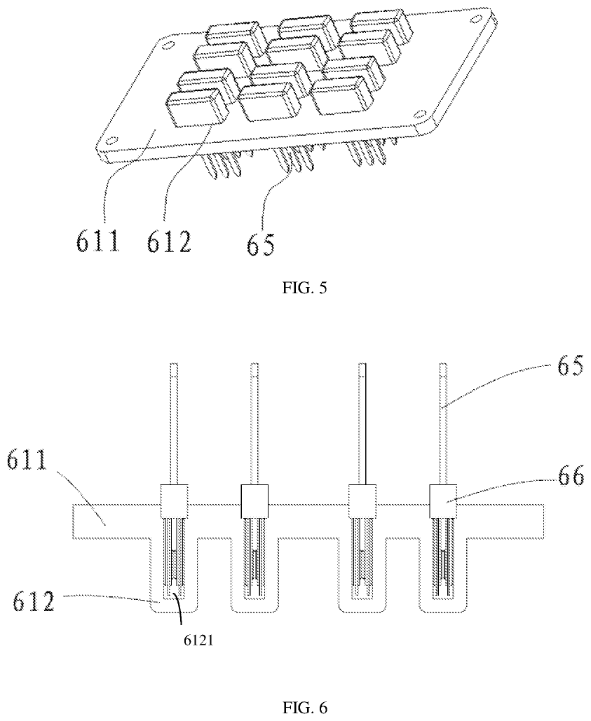

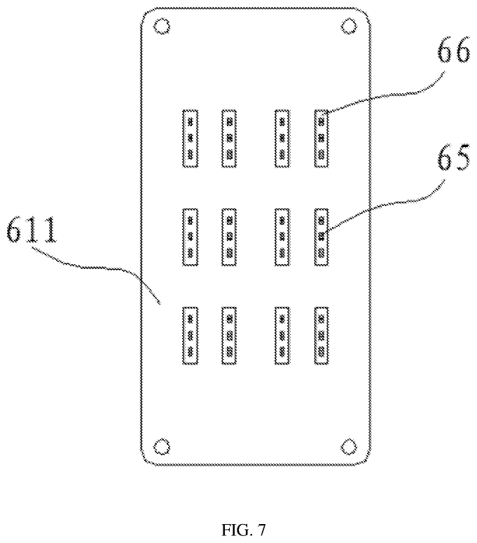

As shown in FIGS. 4-8, the IGBT package heat dissipation structure includes a heat dissipation substrate body 611 and heat dissipation fins 612 and electrodes 65 respectively fixed on two sides of the heat dissipation substrate body 611. Each heat dissipation fin 612 includes an inner cavity 6121, and a wafer, a first direct bonding copper (DBC) layer 631 and a second DBC layer 632 are arranged in the inner cavity 6121. A left side surface of the wafer 64 is connected to a copper layer on a right surface of the first DBC layer 631 by a second welding layer 622, and a right side surface of the wafer 64 is connected to a copper layer on a left surface of the second DBC layer 632 by a third welding layer 623. The copper layer on the left surface of the first DBC layer 631 is connected to a left surface of the inner cavity 6121 of the heat dissipation fin by a first welding layer 621, and the copper layer on the right surface of the second DBC layer 632 is connected to a right surface of the inner cavity 6121 of the heat dissipation fin by a fourth welding layer 624.

The heat dissipation substrate body 611 is provided with an insulating housing 66 at a position corresponding to the inner cavity 6121 of each heat dissipation fin 612, and the insulating housing 66 serves as a support member for supporting an electrode 65, and meanwhile, the insulating housing 66 insulates the electrode 65 from the heat dissipation substrate body 611. The electrode 65 is extended through the insulating housing 66 and enters the inner cavity of the heat dissipation fin 612. A lower end of the electrode 65 is connected with the copper layer on the right surface of the first DBC layer 631 and the copper layer on the left surface of the second DBC layer 632 to realize the input of current to the wafer 64 and output of the current from the wafer 64. The inner cavity 6121 of the heat dissipation fin 612 is also filled with heat conduction silica gel or other heat dissipation materials, so that the heat dissipation effect of the IGBT module 6 is enhanced.

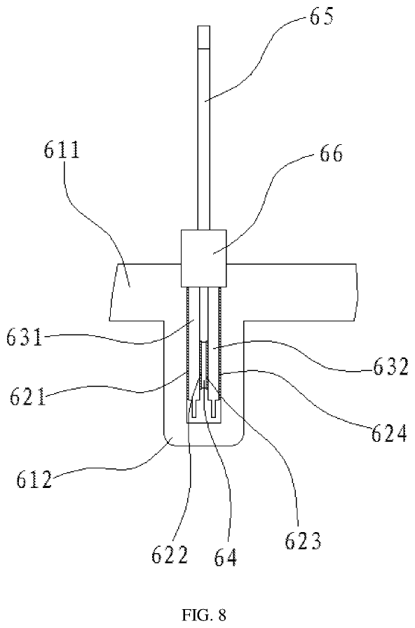

The heat dissipation fin 612 of the present embodiment is not limited to that shown in FIG. 8, for example, in FIG. 8, the inner cavity 6121 of the heat dissipation fin 612 is an upside down H shape with the bottom closed, the inner cavity 6121 of the heat dissipation fin 612 may be a U-shaped cavity or a trapezoidal cavity, and the bottom of the inner cavity 6121 may not be closed.

The above embodiments are only for illustrating the technical concept and features of the present application, and the purpose thereof is to enable a person skilled in the art to understand the contents of the present application and to implement the present application, and not to limit the scope of the present application. All modifications made according to the spirit of the main technical solution of the present application shall fall within the claimed scope of the present application.

What is claimed is:

1. An insulated gate bipolar transistor (IGBT) package heat dissipation structure, comprising a heat dissipation substrate body, heat dissipation fins and electrodes respectively fixed on two sides of the heat dissipation substrate body;
wherein each heat dissipation fin is provided with an inner cavity, a wafer, a first direct bonding copper (DBC) layer and a second DBC layer are arranged in the inner cavity; an electrode is extended through the heat dissipation substrate body, and connected with the wafer through the first DBC layer and the second DBC layer.

2. The IGBT package heat dissipation structure according to claim 1, wherein a left side surface of the wafer is connected with a copper layer on a right surface of the first DBC layer by a second welding layer, and a right side surface of the wafer is connected with a copper layer on a left surface of the second DBC layer by a third welding layer.

3. The IGBT package heat dissipation structure according to claim 2, wherein the electrode is connected to the copper layer on the right surface of the first DBC layer and the copper layer on the left surface of the second DBC layer to connect with the wafer to supply current to the wafer.

4. The IGBT package heat dissipation structure according to claim 3, wherein a copper layer on a left surface of the first DBC layer is connected with a left surface of the inner cavity of the heat dissipation fin by a first welding layer, and a copper layer on a right surface of the second DBC layer is connected with a right surface of the inner cavity of the heat dissipation fin by a fourth welding layer.

5. The IGBT package heat dissipation structure according to claim 3, wherein the heat dissipation substrate body is provided with an insulating housing at a position corresponding to the heat dissipation fin, and the electrode is extended into the inner cavity of the heat dissipation fin through the insulating housing.

6. The IGBT package heat dissipation structure according to claim 5, wherein the inner cavity of the heat dissipation fin is filled with a heat dissipation material.

7. The IGBT package heat dissipation structure according to claim 1, wherein the inner cavity of the heat dissipation fin is an upside down H shape, a U shape, or a trapezoidal shape with a bottom closed.

8. The IGBT package heat dissipation structure according to claim 1, wherein a bottom of the inner cavity of the heat dissipation fin is not closed.

9. A motor controller, comprising a main housing and a film capacitor, a printed circuit board (PCB), a three-phase output module, an IGBT module, and a bus module passing through the main housing,
wherein the IGBT module comprises the IGBT package heat dissipation structure according to claim 1, and the heat dissipation substrate body is shared by a plurality of IGBT package heat dissipation structures.

10. The motor controller according to claim 9, wherein a water channel for heat dissipation is provided in the main housing, and the heat dissipation fins of the IGBT module are disposed in the water channel.
